(19) United States
(12) Patent Application Publication
(10) Pub. No.: US 2011/0076382 A1
(43) Pub. Date: Mar. 31, 2011

(54) AQUEOUS PLANT PROTEIN PREPARATION AND METHOD FOR PRODUCING THE SAME

(75) Inventors: Klaus Müller, Freising (DE); Peter Eisner, Freising (DE); Michael Schott, Langenbach (DE); Jürgen Bez, Munchen (DE); Stefanie Bader, Freising (DE); Katrin Hasenkopf, Freising (DE); Michael Frankl, Munchen (DE)

(73) Assignee: Fraunhofer-Gesellschaft zur Forderung der angewandten Forschung e.V., Munchen (DE)

(21) Appl. No.: 12/449,074

(22) PCT Filed: Jan. 22, 2008
(86) PCT No.: PCT/DE2008/000112

§ 371 Date: Jul. 22, 2009

(30) Foreign Application Priority Data
Jan. 23, 2007 (DE) 10 2007 003 263.5

(51) Int. Cl. A23J 3/00; C07K 14/415; A23L 1/305; A23J 3/14; B65D 85/00; B65B 63/08

(52) U.S. Cl. 426/656; 530/370; 426/442; 206/525; 53/440

(57) ABSTRACT

The present invention relates to a plant protein preparation, preferably from lupine protein, and a method for producing the same. The protein preparation consists of an aqueous mixture with a water content >75% by weight and a solid material mass, which contains over 70% by weight of plant proteins, wherein a proportion of the plant proteins are denatured. The preparation has excellent techno-functional properties, is easy to process and displays large-scale stability to fat oxidation. It can be used as an ingredient for producing foodstuffs and as a substitute for commercial plant and animal protein preparations.

### TECHNICAL FIELD

The invention relates to an aqueous plant protein preparation, preferably a lupine protein, a method for producing the same and also the use of the preparation in foodstuffs.

### BACKGROUND ART

The use of plant proteins as an ingredient for producing foodstuffs as a substitute for animal raw materials such as egg or milk protein is becoming increasingly important. Plant proteins display excellent techno-functional properties for the development of texture in a large number of food applications. Protein preparations made from raw materials such as soya, rice, wheat, peas, lupines or other protein-containing plant seeds are used in foodstuffs as water binders, oil binders, gel-forming agents, emulsifiers or foaming agents, for example.

In order to largely eliminate microbiological deterioration, state-of-the-art protein preparations are supplied on the market in dry, powder form. There are known flours with a protein content <50%, concentrates with a protein content >60% and isolates with a protein content >90%. Powdered ingredients usually have a particle size of under 300 μm and tend to form agglomerates when stirred into water, which is undesirable and takes a long time to stir in.

In order to avoid fat oxidation, in other words, rancidification, defatted raw materials are preferably used in the production of protein preparations. The protein preparations thereby obtained have a fat content of under 2% (method of analysis: Soxhlet) and therefore only create small quantities of fat oxidation products during storage. The disadvantage of large-scale oil separation lies in the high cost of the process. Expensive chemicals such as hexane are needed and high costs are associated with equipping the plant for explosion-proof operation.

Another possibility for reducing the rancidification of protein products, even with fat contents greater than 2%, is offered by thermal treatment or toasting. This involves the whole or crushed seed being heated either dry or moist partly under pressure to temperatures of over 120° C. in some cases, which deactivates the seed's own enzymes and prevents subsequent enzyme-catalysed fat oxidation reactions.

However, the dry or moist thermal treatment causes considerable damage to the proteins and therefore a decline in the techno-functionality of the preparations. The preparations do not readily dissolve in water or in the foodstuff matrix, resulting in a grainy feel in the mouth. Consequently, toasted preparations only display a satisfactory techno-functional profile for lower-grade applications.

The object of the present invention is to provide a plant protein preparation that has excellent techno-functional properties, is easy to process and displays large-scale stability to fat oxidation. The preparation is to be used as an ingredient for the production of foodstuffs and as a substitute for commercial plant and animal protein preparations.

### DESCRIPTION OF THE INVENTION

The object is solved by the Protein preparation and the process described in claims 1 and 7. Advantageous embodiments of the preparation and of the process are the subject-matter of the dependent claims or can be inferred from the following description.

Unlike traditional protein preparations, the protein preparation according to the invention has a water content of over 75% by weight. The solid material in the product contains more than 70% by weight of plant protein, particularly advantageously >80% by weight. Only a small proportion of ≧25% (in relation to proteins) of the proteins in the preparation according to the invention are denatured. This means that some of the proteins have an unfolded structure. This denatured or partly denatured structure may, for example, be achieved by exposure to a temperature of >65° C. for a period of 0.1 to 15 minutes. The combination of high temperature and time must be selected in such a way as to avoid greater denaturation. For the same reason, extreme pH values of <3 or >9 must also be avoided. The residual part of the product's dry substance, possibly with the exception of insoluble fibre, advantageously contains fat or oil from the seed. The proportion of insoluble fibre is preferably below 10% by weight relative to the dry substance. This means that any impairment of the way it feels in the mouth on account of insoluble fibres is largely excluded.

In the present application, the protein content as % by weight is determined by analysing the nitrogen content as % by weight and multiplying this value by a factor of 6.25.

The protein preparation according to the invention is in the form of an aqueous mixture, particularly a homogeneous protein suspension or a solution. The viscosity of this protein preparation at a shear rate of 300 l/s and a temperature of 20° C. is advantageously over 10 mPas.

The protein preparation according to the invention has a series of advantages over the dry, powdered preparations available on the market. The product is very easily and almost completely soluble in water or foodstuffs containing water or it can be stirred into water very simply and uniformly. There are none of the wetting problems that occur when using powder preparations. This means it can be processed quickly and easily in the food industry. As a result, the production of a protein base suspension of water and protein powder that had hitherto been necessary can be dispensed with, which saves on production time. The simple wetting and even mixing of the preparation according to the invention with the foodstuff matrix mean that the product has an improved feeling in the mouth.

The high protein content in the dry substance means that sensitive foodstuffs, which preclude the use of fibrous ingredients, such as mayonnaise, for example, can also be produced.

The suggested protein preparation occurs in an advantageous embodiment as a solution or a homogeneous suspension with a viscosity of over 10 mPas. In relation to consistency, it therefore only differs slightly from fresh egg yolk or homogenised full egg. The consequence of this is that no adjustments need be made to the process compared with the processing of egg. A further advantage is that the preparation can be supplied to the foodstuff manufacturer ready for processing. This means that the preparation can be processed directly without preparing a preliminary suspension, such as liquid egg.

The high protein content of the dry substance combined with the partial denaturation of the protein means that the preparation has exceptionally good techno-functional properties. Consequently, when lupine protein is used in the preparation, for instance, the preparation displays values, particularly in the emulsifier capacity area, that are in some cases 100% higher than the values of commercial powder protein preparations made from lupine or soya. Consequently, the quality of the partly denatured plant protein present in the preparation is significantly higher, compared with commercial plant proteins.

A further advantage lies in the possibility of slowing down considerably the rancidification of the plant oil or fat contained in the preparation. This may be achieved by cooling or deep-freezing. In the frozen state the preparation can be stored for many months without the sensory properties being affected by fat oxidation products. Surprisingly, the preparation's stability at temperatures below freezing is particularly demonstrated when it has a high water content. If dry, powder preparations are stored at temperatures around or below 0° C., unlike the water-containing preparation in the invention, the fat oxidation in these preparations is partially accelerated compared with a storage temperature of +15° C.

Consequently, the protein preparation according to the invention displays particular benefits in relation to storage stability. Microbial storage stability is also guaranteed by deep-freezing.

This also demonstrates that deep-freezing the protein preparation according to the invention does not have a negative effect on techno-functionality. Hence, in tests using a frozen lupine protein with an 80% water content after a 12-month storage period, the same emulsifying capacity could still be measured as prior to deep-freezing.

Production of the protein preparation according to the invention may involve the following process stages:
1) Crushing the seeds of the protein plants used as the raw material, e.g. by grinding or flaking, having shelled them beforehand where necessary.
2) Pre-extracting the crushed or flaked seeds in 5 to 10 times the volume of water 1-3 times at acid pH values, preferably close to the isoelectric point (pH <5) and/or at temperatures <25° C., and separating the solid material from the liquid phase mechanically after each pre-extraction stage.
3) Extracting the plant protein from the pre-extracted solid material with water at a pH value >6.5, preferably >7.0, and then separating the insoluble elements, such as fibres, for example, mechanically.
4) Precipitating the dissolved protein from the solution by adding acid, preferably close to the isoelectric point.
5) Separating the precipitated protein from the supernatant by solid/liquid separation according to the state of the art. The moist protein preparation precipitated and separated from the supernatant contains approx. 20 to 25% dry mass, for example.
6) Only optional: Neutralising the precipitated protein and adjusting the dry substance content, e.g. to 15% by weight by adding water.
7) Heating the precipitated protein to >65° C.

The preparation then has a composition according to the invention and can be packed and if necessary cooled or deep-frozen.

It is also possible before or during production of the preparation according to the invention for additional heating to temperatures >95° C. to take place. Special effects in relation to the preparation's flavour or colouring can then be achieved if necessary.

The preparation is preferably used as a substitute for milk protein or egg protein or dry plant protein in foodstuffs. The similarity in consistency to liquid egg yolk or liquid whole egg means that these food ingredients can be particularly effectively substituted by the preparation according to the invention.

The preparation according to the invention can be used in foodstuff emulsions, such as soups, sauces, puddings, desserts, spreads, mayonnaise, chocolate fillings or baking. It may also be used as an additive to give texture or bind fat or water in sausages and salamis, pasta, baking or patisserie.

The preparation according to the invention contains plant proteins made from a raw material or protein mixtures made from several raw materials. Oil seeds such as rape, sunflower seeds, flax seeds and other oil seeds may be used as raw materials, for instance. Proteins from legumes may also be contained in the preparation according to the invention. Examples are soya, peas, lupines, field beans or others. Additional plant raw materials, such as corn, rice or potatoes are also possible.

The use of lupine proteins offers particular advantages. Hence, if the protein content of the preparation according to the invention is made up of lupine protein, apart from its techno-functionality, it also has an outstanding bioactive potential for cholesterine adjustment in humans. This can be used in the production of functional food products.

Particular benefits arise for the marketing of the preparation according to the invention. It can be very efficiently packed using the same packaging formats and materials as are state-of-the-art for milk products and liquid egg products. Examples are packaging formats such as composite packaging (e.g. Tetrapak®), tubular bags, stand-up pouches, deep-drawn plastic tubs and vacuum packs. This enables the same packing machines to be used for the preparation as are known from dairy engineering, which saves on costs. The sterile or aseptic decanting known in the state of the art after pasteurisation, sterilisation or UHT heating enables the preparation to be kept for several weeks in a cooled state, without the preparation having to be deep-frozen. It is also possible for the preparation to be heated after it has been decanted into the packaging, e.g. in a water bath, a tunnel pasteuriser or an autoclave to produce a sort of canned product, which means the minimum shelf life can be further increased. It is shown here that at temperatures of over 110° C. the emulsifying capacity can be increased still further in the packaging too.

The possibility of using packaging formats such as Tetrapak® or other milk product packaging makes for ease of storage and measuring for the user in the food industry. For larger production operations, the same packing drums, containers and presentational forms as are known from the industrial-scale transportation of liquid egg can be used.

The production of a protein preparation according to the invention made from lupine proteins is once again briefly described below using an exemplary embodiment.

10 kg lupine seeds are shelled in a lower-course shelling unit. The shells are then air-separated from the fleshy kernel. The shell-free kernels are flaked using a flaking roller into 0.2 mm thick flakes. The lupine flakes thereby obtained are dispersed in an impeller-type mixer with 10 parts water with a pH value of 4.5 at a temperature of 15° C. The starting mixture is kept at a constant pH of 4.5 and 15° C. for 60 minutes. The aqueous phase is then separated from the insoluble, fibre fraction in a decanter.

The moist solid material is once again dispersed in an impeller-type mixer with 5 parts water (pH value 7.5, temperature 35° C.). After 45 minutes, the starting mixture that has been kept constant at pH 7.5 and 35° C. is separated into an insoluble solid material and a protein-containing extract by decantation. The protein contained in the extract is adjusted to a pH value of 4.5 by adding hydrochloric acid, thereby precipitated and separated from the aqueous supernatant by separators. The precipitated protein is pasteurised by heating for 3 minutes in a tubular heat exchanger to 72° C. and decanted into 5 kg tubular bags and cooled to 5° C.

The low denaturation desired is hereby achieved by the low temperatures selected or the limited time at a higher temperature and the pH values chosen.

1-11. (canceled)

12. A plant protein preparation present as an aqueous mixture, comprising an aqueous mixture having a water content greater than 75% by weight and a solid material mass containing over 70% by weight of plant proteins, wherein a proportion less than or equal to 25% of the plant proteins are denatured.

13. The plant protein preparation according to claim 12, wherein a residual proportion of the solid material mass contains plant fat or plant oil.

14. The plant protein preparation according to claim 12, wherein a proportion of insoluble fiber in the aqueous mixture is less than 10% by weight relative to the solid material mass.

15. The plant protein preparation according to claim 13, wherein a proportion of insoluble fiber in the aqueous mixture is less than 10% by weight relative to the solid material mass.

16. The plant protein preparation according to claim 12, wherein the solid material mass contains greater than 80% by weight plant proteins.

17. The plant protein preparation according to claim 12, wherein the aqueous mixture has a viscosity of greater than 10 mPas at a shear rate of 300 l/s and a temperature of 20° C.

18. The plant protein preparation according to claim 12, in combination with, and present in, sealed, liquid-tight packaging.

19. A method for producing a plant protein preparation according to claim 12, 16 or 17, comprising
(a) crushing seeds of protein-containing plants to provide crushed seeds;
(b) pre-extracting a solid material portion from the crushed seeds by rinsing the crushed seeds in water at least once at an acid pH value and mechanically separating the solid material portion from a liquid phase provided during said pre-extracting in each rinsing;
(c) extracting plant proteins from the solid material portion obtained in (b) with water at a pH value greater then 6.5, at which the plant proteins dissolve in the water, and mechanically separating insoluble elements to provide dissolved plant proteins in solution;
(d) precipitating the dissolved plant proteins from the solution by adding acid;
(e) separating the plant proteins precipitated in (d) by solid/liquid separation, wherein a proportion greater than 70% by weight of plant proteins is obtained in a solid material mass;
(f) heating the solid material mass, to pasteurize the plant proteins, to a temperature greater than 65° C. for a period at which a predetermined denaturation level of less than or equal to 25% is not exceeded; and
(g) adding water to form an aqueous mixture before or after said heating, wherein the aqueous mixture is adjusted to a dry substance content of less than 25% by weight through the adding of water.

20. The method according to claim 19, wherein said pre-extracting takes place at a water pH value of less than 5.

21. The method according to claim 19, wherein said extracting takes place at a water pH value of less than 7.0.

22. The method according to claim 20, wherein said extracting takes place at a water pH value of less than 7.0.

23. The method according to claim 19, further comprising transferring the aqueous mixture into packaging, sealing the packaging so that the packaging is liquid-tight, and heating the aqueous mixture in the packaging following said sealing to a temperature greater than 110° C.

24. The plant protein preparation according to claim 12, present as a substitute for milk protein, egg protein or dry plant protein in a foodstuff.

25. The method according to claim 19, further comprising incorporating the protein preparation into a foodstuff as a substitute for a milk protein, egg protein or dry plant protein.

26. The plant protein preparation of claim 12, wherein the plant proteins include lupine proteins.
